(19) United States
(12) Patent Application Publication
(10) Pub. No.: US 2008/0058923 A1
(43) Pub. Date: Mar. 6, 2008

(54) BIOCORRODIBLE METALLIC IMPLANT HAVING A COATING OR CAVITY FILLING MADE OF GELATIN

(75) Inventors: Torben Bertsch, Nuernberg (DE); Alexander Borck, Aurachtal (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/850,346

(22) Filed: Sep. 5, 2007

(30) Foreign Application Priority Data
Sep. 6, 2006 (DE) 10 2006 042 313.5

(51) Int. Cl. A61F 2/06

(52) U.S. Cl. 623/1.46

(57) ABSTRACT

An implant made of a biocorrodible metallic material and having a coating or cavity filling comprising gelatin.

### PRIORITY CLAIM

This patent application claims priority to German Patent Application No. 10 2006 042 313.5, filed Sep. 6, 2006, the disclosure of which is incorporated herein by reference in its entirety.

### FIELD

The present disclosure relates to implants made of biocorrodible metallic material, and having a coating or cavity filling, and a method for coating an implant or filling a cavity of an implant.

### BACKGROUND

Implants are used in modern medical technology in manifold embodiments. Implants are used, for example, for supporting vessels, hollow organs, and duct systems (endovascular implants), for attachment to and temporary fixing of tissue implants and tissue transplants, and for orthopedic purposes, for example, as nails, plates, or screws.

Thus, for example, the implantation of stents has been established as one of the most effective therapeutic measures in the treatment of vascular illnesses. Stents provide a support function in the hollow organs of a patient. Stents of typical construction have a filigree support structure made of metallic struts for this purpose, which is first provided in a compressed form for introduction into the body and is expanded at the location of application. One of the main areas of application of such stents is permanently or temporarily expanding and keeping open vascular constrictions, in particular, constrictions (stenoses) of the coronary vessels. In addition, for example, aneurysm stents are also known which are used to support damaged vascular walls.

Stents have a peripheral wall of sufficient supporting force to keep the constricted vessel open to the desired degree and a tubular main body through which the blood flow continues to run unimpeded. The supporting peripheral wall is frequently implemented as a latticed structure, which allows the stent to be inserted in a compressed state having a small external diameter up to the constriction point of the particular vessel to be treated and to be expanded there with the aid of a balloon catheter, for example, enough that the vessel has the desired, enlarged internal diameter. To avoid unnecessary vascular damage, the stent should not elastically recoil at all or, in any case, should elastically recoil only slightly after the expansion and removal of the balloon, so that the stent only has to be expanded slightly beyond the desired final diameter upon expansion. Further criteria which are desirable in a stent include, for example, uniform area coverage and a structure which allows a certain flexibility in relation to the longitudinal axis of the stent. In practice, the stent is typically molded from a metallic material to implement the cited metallic properties.

In addition to the mechanical properties of a stent, the stent should comprise a biocompatible material to avoid rejection reactions. Currently, stents are used in approximately 70% of all percutaneous interventions; however, an in-stent restenosis occurs in 25% of all cases because of excess neointimal growth, which is caused by a strong proliferation of the arterial smooth muscle cells and a chronic inflammation reaction. Various solution approaches are followed to reduce the restenosis rate.

One approach for reducing the restenosis rate includes providing a pharmaceutically active substance (active ingredient) on the stent, which counteracts the mechanisms of restenosis and supports the course of healing. The active ingredient is applied in pure form or embedded in a carrier matrix as a coating or filled in cavities of the implant. Examples comprise the active ingredients sirolimus and paclitaxel.

A further, more promising approach for solving the problem is the use of biocorrodible materials and their alloys because, typically, a permanent support function by the stent is not necessary; the initially damaged body tissue regenerates. Thus, it is suggested in German Patent Application No. 197 31 021 A1 that medical implants be molded from a metallic material whose main component is iron, zinc, or aluminum or an element from the group consisting of alkali metals or alkaline earth metals. Alloys based on magnesium, iron, and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc, and iron. Furthermore, the use of a biocorrodible magnesium alloy having a proportion of magnesium >90%, yttrium 3.7-5.5%, rare earth metals 1.5-4.4%, and the remainder <1% is known from German Patent Application No. 102 53 634 A1, which is suitable, in particular, for producing an endoprosthesis, e.g., in the form of a self-expanding or balloon-expandable stent. The use of biocorrodible metallic materials in implants may result in a significant reduction of rejection or inflammation reactions.

The combination of active ingredient release and biocorrodible metallic material appears particularly promising. The active ingredient is applied as a coating or introduced into a cavity in an implant, usually embedded in a carrier matrix. For example, stents made of a biocorrodible magnesium alloy having a coating made of a poly(L-lactide) are known in the art. However, the following problems are still to be solved, in spite of the progress achieved.

The degradation products of the carrier matrix should not have any noticeable influence on the local pH value to avoid undesired tissue reactions, on one hand, and to reduce the influence on the corrosion process of the metallic implant material, on the other hand. Furthermore, the degradation of the carrier matrix should occur more rapidly than the degradation of the main body to avoid undesired interactions of the two processes.

### SUMMARY

The present disclosure describes several exemplary embodiments of the present invention.

One aspect of the present disclosure provides an implant made of a biocorrodible metallic material, the implant having a coating or cavity filling comprising gelatin.

Another aspect of the present disclosure provides a method for coating a stent made of a biocorrodible metallic material, comprising a) producing a coating comprising a gelatin; and b) coating the stent with the gelatin coating.

A further aspect of the present disclosure provides a method for filling a cavity in a stent made of a biocorrodible metallic material, comprising a) producing a filling comprising gelatin; and b) filling the cavity with the filling.

### DETAILED DESCRIPTION

A first aspect of the present disclosure provides an implant made of a biocorrodible metallic material having a coating or cavity filling comprising gelatin.

Gelatin is a mixture of polypeptides having molar masses of approximately 13,500 to 500,000 g/mole (determined by SDS gel electrophoresis or gel chromatography) depending on how it is obtained, which is obtained by hydrolysis of collagen performed to different extents. The amino acid composition largely corresponds to that of collagen, from which it is obtained, and comprises all essential amino acids with the exception of tryptophan and methionine; the main amino acid is hydroxyproline. Gelatin contains 84 to 90 wt.-% (weight-percent) protein and 2 to 4 wt.-% mineral materials; the remainder comprises water. Gelatin is odorless and practically colorless, insoluble in ethanol, ethers, and ketones, but soluble in ethylene glycol, glycerol, formamide, and acetic acid. One differs between two methods of production: the acid method for gelatin of type A and the alkaline method for gelatin of type B. The raw material for gelatin of type A (predominantly pig skin) is subjected to a three-day digestion process. In the production of gelatin of type B, beef split layer (middle layer between leather and the hypodermis) and/or bones are treated for 10-20 days using alkali. The strength of the gelatinous mass is determined using a gelometer (texture analyzer) and specified as the Bloom number. The isoelectric point of gelatin is at pH 7.5 to 9.3 (type A) or 4.7 to 5.2 (type B).

Gelatin may be chemically modified and its properties may be varied widely by reaction of the amino groups above all with monofunctional or polyfunctional reagents such as acylation agents, aldehydes, epoxides, halogen compounds, cyanamide, or activated unsaturated compounds. For purposes of the present disclosure, the term “gelatin” includes the gelatin derivatives obtained as described above.

In pharmacy and medicine, gelatin is used for producing soft and hard capsules, suppositories, as a binder and pressing aid for tablets, as a stabilizer for emulsions and as a blood plasma extender. In cross-linked form, gelatin is used for producing sterile, hemostatic sponges for surgical purposes; in cosmetics as a component of salves, pastes, and creams; as a protective colloid in shampoos, washing and cleaning agents; and in gels having good skin compatibility.

When gelatin is used in the body, neither gelatin nor its degradation products display a noticeable effect on the local pH value. A carrier matrix made of polylactide, in contrast, hydrolizes while forming acid functions which have been held responsible for tissue reactions, such as inflammation. In addition to the positive influence on the surrounding tissue, the influence of the adducts on the degradation of the main body is negligible, in particular, if the main body comprises magnesium and its alloys, i.e., the degradation of the main body is not additionally accelerated by the presence of the adducts.

For purposes of the present disclosure, a coating is an at least partial application of the components onto the main body of the implant, in particular, a stent. Preferably, the entire surface of the main body of the implant or stent is covered by the coating. Alternatively, the gelatin may be provided in a cavity of the implant or stent. The coating or cavity filling comprises gelatin. The weight proportion of gelatin in the components of the coating or cavity filling forming the carrier matrix is at least 30%, preferably at least 50%, especially preferably at least 70%. The components of the coating comprise the materials acting as a carrier matrix, i.e., materials which are necessary for the functional properties of the carrier matrix, e.g., also auxiliary materials for improving the viscosity properties, gel formation, and processability. These components do not comprise the possibly added active ingredient or marker materials.

The gelatin used according to the present disclosure is highly biocompatible and biodegradable. The processing may be performed according to standard methods known in the art.

Gelatin is suitable as a carrier material for absorbing active ingredients, in particular, proteins having a mean molecular weight in the range from 5,000 g/mole to 300,000 g/mole, especially preferably in the range from 40,000 g/mole to 150,000 g/mole. If the molecular weight of the protein is below the specified boundary, a diffusion speed of the protein from the hydrogel provided in use is too large for most local therapeutic applications. In contrast, if the molecular weight of the protein is above the specified highest value, the diffusion speed is too low for the same reason. Furthermore, the gelatin is particularly suitable for absorbing dODNs, antibodies, flavopiridol, and amlodipine. For processing, the gelatin is liquefied by heating, e.g., using microwaves, and the active ingredients are suspended or dissolved. The addition is to be performed before the gelling, i.e., implementation of a hydrogel.

Alternatively or additionally, the gelatin may be used as a carrier matrix for x-ray markers or magnetic resonance markers. The x-ray markers may not be applied directly to the product in implants made of a biocorrodible metallic material, because they would influence the degradation of the stent by forming local elements. In contrast, they are shielded from the main body in the matrix made of gelatin.

The gelatin may be combined with further materials used as the carrier matrix, such as polylactide, for example, to optimize the material properties for the desired intended purposes. It is also conceivable in this context that the carrier matrix has a layered structure, e.g., a base layer made of gelatin and a cover layer made of a further material.

The preparation of the gelatin for the coating/filling of cavities may be performed in buffered solutions. The gelatin is preferably provided as 0.5 to 20 wt.-%, in particular 2 to 20 wt.-% solution in water or a buffered aqueous medium. The solutions may be processed especially simply. The pH value of the solutions is preferably in the range from 5 to 8 to avoid hydrolysis of the gelatin during processing, which would result in lower gel strength.

For purposes of the present disclosure, the term “biocorrodible” refers to metallic materials in which degradation occurs in physiological surroundings which finally results in the entire implant or the part of the implant made of the material losing its mechanical integrity. For purposes of the present disclosure, biocorrodible metallic materials particularly comprise metals and alloys selected from the group consisting of the elements iron, tungsten, and magnesium. For purposes of the present disclosure, an alloy is a metallic microstructure, whose main component is magnesium, iron, or tungsten. The main component is the alloy component whose weight proportion in the alloy is highest. A proportion of the main component is preferably more than 50 wt.-%, in particular more than 70 wt.-%.

The biocorrodible material is preferably a magnesium alloy. In particular, the biocorrodible magnesium alloy contains yttrium and further rare earth metals, because an alloy of this type is distinguished on the basis of its physiochemical properties and high biocompatibility, in particular, its degradation products. Biodegradable magnesium alloys usually have a relatively high degradation speed and, if a carrier matrix having a lower degradation speed in comparison is used, undesired interactions between the two degradation processes may occur. However, these interactions are to be avoided as much as possible in view of the therapeutic requirements. It has been shown that gelatin apparently has a higher degradation speed than the currently typical magnesium alloys, so that the complications noted may be avoided.

A magnesium alloy of the composition rare earth metals 5.2-9.9 wt.-%, yttrium 3.7-5.5 wt.-%, and the remainder <1 wt.-% is especially preferable, magnesium making up the proportion of the alloy to 100 wt.-%. This magnesium alloy has already confirmed its special suitability experimentally and in initial clinical trials, i.e., the magnesium alloy displays a high biocompatibility, favorable processing properties, good mechanical characteristics, and corrosion behavior adequate for the intended uses. For purposes of the present disclosure, the collective term “rare earth metals” include scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The alloys of the elements magnesium, iron, or tungsten are to be selected in the composition in such a way that they are biocorrodible. Artificial plasma, as has been previously described according to EN ISO 10993-15:2000 for biocorrosion assays (composition NaCl 6.8 g/l, CaCl2 0.2 g/l, KCl 0.4 g/l, MgSO4 0.1 g/l, NaHCO3 2.2 g/l, Na2HPO4 0.126 g/l, NaH2PO4 0.026 g/l), is used as a testing medium for testing the corrosion behavior of an alloy under consideration. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces in a way known in the art. The artificial plasma according to EN ISO 10993-15:2000 corresponds to a medium similar to blood and thus represents a possibility for simulating a reproducible physiological environment.

For purposes of the present disclosure, implants are devices introduced into the body via a surgical method and comprise fasteners for bones, such as screws, plates, or nails, surgical suture material, intestinal clamps, vascular clips, prostheses in the area of the hard and soft tissue, and anchoring elements for electrodes, in particular, of pacemakers or defibrillators.

The implant is preferably a stent. Stents of typical construction have a filigree support structure made of metallic struts which is initially provided in an unexpanded state for introduction into the body and is then widened into an expanded state at the location of application. Because of the type of use, brittle coating systems are unsuitable; in contrast, gelatin has particularly suitable material properties, such as viscosity and flexibility sufficient for the purpose. The stent may be coated before or after being crimped onto a balloon.

A second aspect of the present disclosure relates to a method for using gelatin as a coating material for a stent made of a biocorrodible metallic material or as a filling for a cavity in a stent made of a biocorrodible metallic material.

Stents made of the biocorrodible magnesium alloy WE43 (97 wt.-% magnesium, 4 wt.-% yttrium, 3 wt.-% rare earth metals besides yttrium) are coated as described hereinbelow.

The magnesium surfaces of the stent are roughened by treatment using an argon plasma to achieve greater adhesion of the active ingredient on the stent surface. Alternatively or additionally, a surface modification, e.g., by silanization using methoxy or epoxy silanes or with the aid of phosphonic acid derivatives, may increase the adhesion capability to the metallic main body.

A 10 wt.-% aqueous solution of gelatin in a phosphate buffer of pH 7 at approximately 50° C. is used. After cooling the solution to approximately 30° C., an aqueous solution or dispersion of an active ingredient is added while stirring. The mixture obtained is sprayed onto the stent and dried for 24 hours at room temperature. Subsequently, the coating is cross-linked by immersion in 1% aqueous glutaraldehyde solution for 3 minutes, then is washed using an aqueous solution buffered to pH 7 and dried.

In the processing of methacrylated gelatin derivatives, a photoinitiator is added to the coating solution, and the stent is exposed after the film application to cross-link the film.

All patents, patent applications and publications referred to herein are incorporated by reference in their entirety.

What is claimed is:

1. An implant made of a biocorrodible metallic material, the implant having a coating or cavity filling comprising gelatin.

2. The implant of claim 1, wherein the biocorrodible metallic material comprises a magnesium alloy.

3. The implant of claim 1, wherein the implant comprises a stent.

4. A method for coating a stent made of a biocorrodible metallic material, comprising:
a) producing a coating comprising a gelatin; and
b) coating the stent with the gelatin coating.

5. A method for filling a cavity in a stent made of a biocorrodible metallic material, comprising:
a) producing a filling comprising gelatin; and
b) filling the cavity with the filling.
